(19) United States
(12) Patent Application Publication
(10) Pub. No.: US 2016/0198977 A1
(43) Pub. Date: Jul. 14, 2016

(54) WEARABLE APPARATUS FOR OBTAINING BIOLOGICAL INFORMATION AND METHOD OF OBTAINING BIOLOGICAL INFORMATION USING THE SAME

(71) Applicant: SAMSUNG ELECTRONICS CO., LTD., Suwon-si (KR)

(72) Inventors: Kunsun EOM, Seoul (KR); Seongho CHO, Gwacheon-si (KR)

(73) Assignee: SAMSUNG ELECTRONICS CO., LTD., Suwon-si (KR)

(21) Appl. No.: 14/749,771

(22) Filed: Jun. 25, 2015

(30) Foreign Application Priority Data
Jan. 12, 2015 (KR) 10-2015-0004453

(51) Int. Cl. A61B 5/053; A61B 5/11; A61B 5/00; A61B 5/021

(52) U.S. Cl. CPC A61B 5/0537; A61B 5/021; A61B 5/0531; A61B 5/1121; A61B 5/6824

(57) ABSTRACT

Provided is a wearable apparatus for obtaining biological information, the wearable apparatus including: a bio signal measuring unit configured to measure a bio signal of a subject; and a position detector configured to detect a position of the subject wearing the wearable apparatus and determine correction factors with regard to the measured bio signal in response to the detected position. Since the biological information of the subject may be analyzed by reflecting the correction factors determined by the position detector to the measured bio signal, the accuracy of analysis results may be increased, and thus, user convenience regarding measuring positions may be increased.

### CROSS-REFERENCE TO RELATED APPLICATION

This application claims priority from Korean Patent Application No. 10-2015-0004453, filed on Jan. 12, 2015 in the Korean Intellectual Property Office, the disclosure of which is incorporated herein in its entirety by reference.

### BACKGROUND

1. Field

Apparatuses and methods consistent with exemplary embodiments relate to a wearable apparatus for obtaining biological information and method of obtaining biological information using the same.

2. Description of the Related Art

With developments in medical science and extended life expectancies, interest in health care and medical devices has increased. Accordingly, various medical devices for use in hospitals and inspection clinics, medium-sized medical devices installed in government agencies, personal small-sized medical devices, and personal mobile healthcare devices have been proposed.

A body composition measurer that is a kind of health care device measures body composition through bioelectrical impedance analysis (BIA). According to BIA, an impedance of a human body is measured by applying a current to a human body which is considered as a combination of impedances and measuring a voltage according to the current, and the body composition such as moisture in the human body, an amount of protein, bones, and fat may be analyzed based on the measured impedance.

A hemadynamometer, which is small, portable, and cuffless in comparison with a large cuffed hemadynamometer, is widely used. Photoplethysmography (PPG) is measured through an optical method, and blood pressure may be measured based on the measured PPG.

### SUMMARY

Exemplary embodiments provide a wearable apparatus for obtaining biological information and method of obtaining biological information using the same.

According to an aspect of an exemplary embodiment, there is provided a wearable apparatus for obtaining biological information, includes: a bio signal measuring unit configured to measure a bio signal of a subject; and a position detector configured to detect a position of the subject wearing the wearable apparatus and determine correction factors with regard to the measured bio signal in response to the detected position.

The position detector may include a circuit for measuring a radio frequency (RF) characteristic, and the position detector may detect the position of the subject from the measured RF response characteristic that changes according to a relative location between the subject wearing the wearable apparatus and an antenna included in the circuit.

The wearable apparatus may further include a wireless communication unit. The position detector may share at least one of circuit components forming the wireless communication unit.

The position detector may include a circuit that measures a bio impedance, and the position detector may detect the position of the subject from the measured bio impedance of the subject wearing the wearable apparatus.

The bio signal measuring unit may include: a first input electrode and a first output electrode arranged to be in contact with one wrist of the subject; a second input electrode and a second output electrode arranged to be in contact with a body part corresponding to the other wrist of the subject; a measuring unit configured to measure a bio impedance of the subject by applying a current to the first and second input electrodes and detecting a voltage from the first and second output electrodes; and an analysis unit configured to analyze a body composition of the subject by reflecting the correction factors determined by the position detector to the bio impedance measured by the measuring unit.

The position detector may be configured to detect at least one of an angle at which an arm of the subject is bent and a distance between the wrists of and a torso of the subject as a measuring position.

The position detector may include a circuit for measuring an RF response characteristic and may be configured to detect a measuring position of the subject from the RF response characteristic that changes according to a relative location between the subject and an antenna included in the circuit.

The position detector may be configured to use data stored by quantifying a degree to which the RF response characteristic changes according to a measuring position of the subject and to detect the measuring position of the subject by comparing the data with the measured RF response characteristic.

The position detector may be configured to use data stored by quantifying a correction factor regarding the measuring position of the subject and may determine the correction factor by comparing the measuring position of the subject with the data stored by quantifying the correction factor.

The position detector may further include one or more RF terminals to be placed on a body part, other than the wrist on which the wearable apparatus is placed.

The antenna may have a circular or oval radiation pattern.

The position detector may be configured to receive information regarding the measuring position of the subject and, quantify a correction factor regarding the measuring position of the subject by using data stored in the wearable apparatus, and determine the correction factor by comparing the received information regarding the measuring position of the subject with the stored data.

The bio signal may indicate a blood pressure of the subject.

According to an aspect of another exemplary embodiment, there is provided a method of obtaining biological information by using the above wearable apparatus, including: wearing, measuring a bio impedance of a subject by applying a current to the first input electrode and the second input electrode and detecting a voltage from the first output electrode and the second output electrode, the first input electrode and the first output electrode being in contact with one wrist of a subject, and the second input electrode and the second output electrode being in contact with a body part of the other wrist; detecting a measuring position of the subject and determining a correction factor regarding the measuring position; and analyzing the body composition of the subject by reflecting the correction factor to the bio impedance measured by the measuring unit.

The method may further include checking whether each of the first input electrode, the second input electrode, the first output electrode, and the second output electrode of the wearable apparatus is placed in contact with the subject.

The measuring of the bio impedance may be performed after the detecting of the measuring position of the subject and the determining the correction factor are performed.

After the measuring of the bio impedance is performed, the detecting of the measuring position of the subject and the determining the correction factor may be further performed.

The detecting of the measuring position of the subject and the determining the correction factor may be performed after the measuring of the bio impedance.

The measuring of the bio impedance, the detecting of the measuring position of the subject, and the determining the correction factor may be respectively repeated at least twice.

The measuring of the bio impedance, the detecting of the measuring position of the subject, and the determining the correction factor may be simultaneously performed.

### BRIEF DESCRIPTION OF THE DRAWINGS

The above and/or other aspects will be more apparent by describing certain exemplary embodiments, with reference to the accompanying drawings, in which:

FIG. 1 is a block diagram of a schematic structure of a wearable apparatus for obtaining biological information according to an exemplary embodiment;

FIG. 2 is a block diagram of an exemplary structure of a measuring unit included in the wearable apparatus for obtaining biological information of FIG. 1;

FIGS. 3A and 3B are perspective views of an exterior of a wearable apparatus for obtaining biological information and respectively illustrate outer and inner surfaces of a strap;

FIG. 4 illustrates a case where a body part comes into contact with an electrode when body composition is measured using a wearable apparatus for obtaining biological information, according to an exemplary embodiment;

FIG. 5 schematically illustrates an equivalent circuit when a body part comes into contact with an electrode as illustrated in FIG. 4;

FIGS. 6A to 6D are examples of a variety of certain positions of a subject when body composition is measured using a wearable apparatus for obtaining biological information, according to an exemplary embodiment;

FIG. 7 is a block diagram of an exemplary structure of a position detector included in a wearable apparatus for obtaining biological information, according to an exemplary embodiment;

FIG. 8 is a graph for explaining a radio frequency (RF) response characteristic that changes in a position detector, depending on a measuring position of a subject;

FIG. 9 illustrates a schematic structure of a wearable apparatus for obtaining biological information according to another exemplary embodiment;

FIG. 10 illustrates an exemplary structure of a circuit that may be used as an RF response characteristic measuring circuit of FIG. 7;

FIG. 11 illustrates an exemplary structure of a circuit that may be used as an RF response characteristic measuring circuit of FIG. 7;

FIG. 12 is a block diagram of an exemplary structure of a position detector included in a wearable apparatus for obtaining biological information according to another exemplary embodiment;

FIG. 13 is a schematic flowchart for explaining a method of measuring body composition according to an exemplary embodiment;

FIG. 14 is a schematic flowchart for explaining a method of measuring body composition according to another exemplary embodiment;

FIG. 15 is a schematic flowchart for explaining a method of measuring body composition according to another exemplary embodiment;

FIG. 16 is a schematic flowchart for explaining a method of measuring body composition according to another exemplary embodiment;

FIG. 17 is a schematic flowchart for explaining a method of measuring body composition according to another exemplary embodiment; and

FIG. 18 is a block diagram of a schematic structure of a wearable apparatus for obtaining biological information according to another exemplary embodiment.

### DETAILED DESCRIPTION

Exemplary embodiments are described in greater detail below with reference to the accompanying drawings.

In the following description, like drawing reference numerals are used for like elements, even in different drawings. The matters defined in the description, such as detailed construction and elements, are provided to assist in a comprehensive understanding of the exemplary embodiments. However, it is apparent that the exemplary embodiments can be practiced without those specifically defined matters. Also, well-known functions or constructions are not described in detail since they would obscure the description with unnecessary detail.

It will be understood that when a component, such as a layer, a film, a region, or a plate, is referred to as being “on” another component, the component can be directly on the other component or intervening components may be present thereon.

While such terms as “first”, “second”, etc., may be used to describe various components, such components must not be limited to the above terms. The above terms are used only to distinguish one component from another.

An expression used in the singular encompasses the expression of the plural, unless it has a clearly different meaning in the context. In the present specification, it is to be understood that terms such as “including”, “having”, and “comprising” are intended to indicate the existence of the features, numbers, steps, actions, components, parts, or combinations thereof disclosed in the specification, and are not intended to preclude the possibility that one or more other features, numbers, steps, actions, components, parts, or combinations thereof may exist or may be added.

Also, in the present specification, it will be understood that a term such as a “unit” is intended to indicate a hardware component such as a processor or a circuit, and/or a software component implemented by a hardware component such as a processor.

Expressions such as “at least one of,” when preceding a list of elements, modify the entire list of elements and do not modify the individual elements of the list.

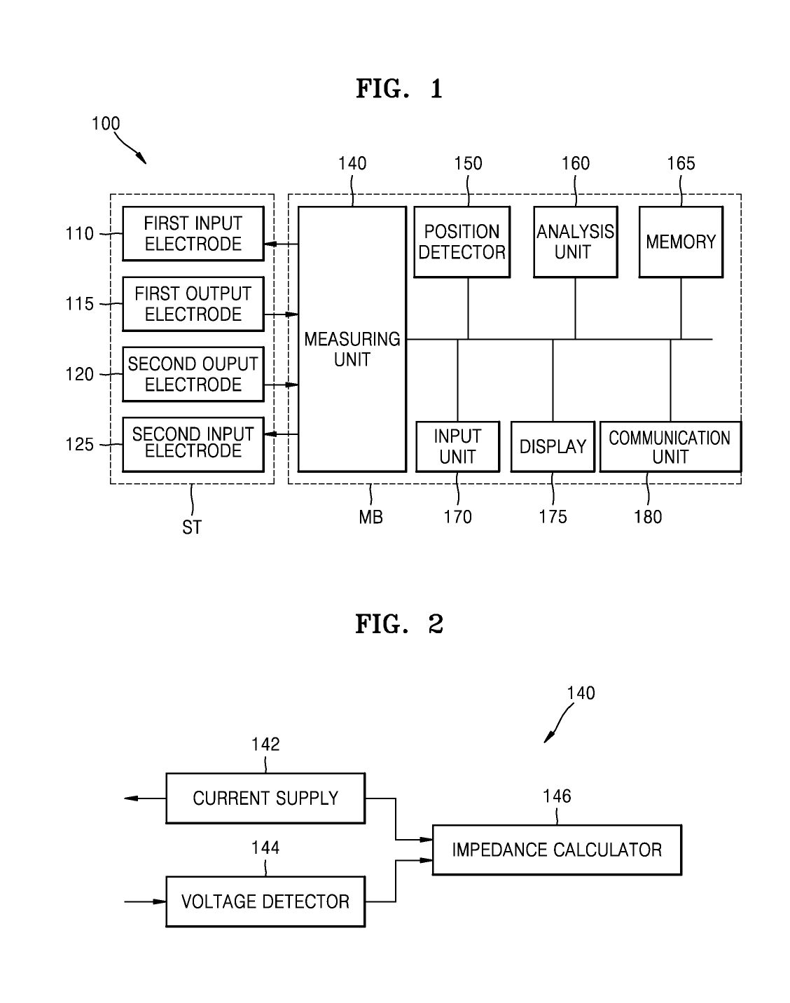

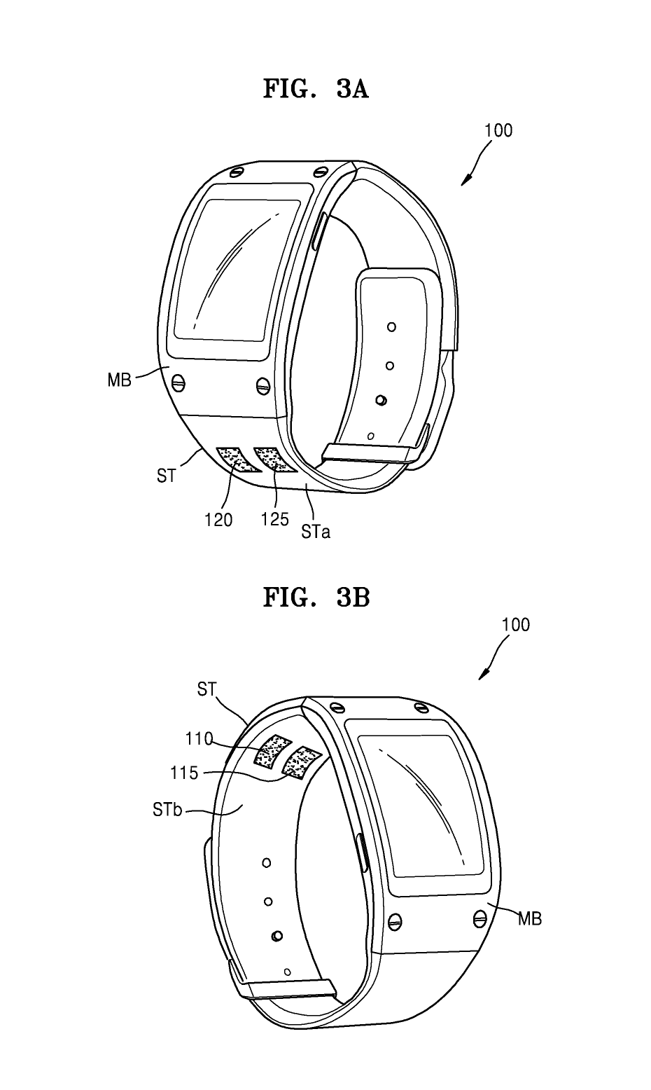

FIG. 1 is a block diagram of a schematic structure of a wearable apparatus 100 for obtaining biological information according to an exemplary embodiment, FIG. 2 is a block diagram of an exemplary structure of a measuring unit 140 included in the wearable apparatus 100 for obtaining biological information of FIG. 1, and FIGS. 3A and 3B are perspective views of an exterior of the wearable apparatus 100 for obtaining biological information and respectively illustrate outer and inner surfaces of a strap.

The wearable apparatus 100 for obtaining biological information includes first and second input electrodes 110 and 125 arranged to be in contact with a body of a subject, first and second output electrodes 115 and 120, a measuring unit 140 configured to measure a bio impedance of the subject by applying a voltage to the first and second input electrodes 110 and 125 and detecting a voltage from the first and second output electrodes 115 and 120, a position detector 150 configured to detect a position of the subject and calculate a correction factor in accordance to the detected position, and an analysis unit 160 configured to analyze body composition by reflecting the correction factor calculated by the position detector 150 to the measured bio impedance.

Also, the wearable apparatus 100 for obtaining biological information may further include a memory 165, an input unit 170, a display 175, and a communication unit 180.

The wearable apparatus 100 for obtaining biological information includes a main body MB and straps ST, As shown in FIG. 1, the first and second input electrodes 110 and 125, and the first and second output electrodes 115 and 120 are arranged on the straps ST, and the measuring unit 140, the position detector 150, the analysis unit 160, the input unit 170, the display 175, and the communication unit 180 are arranged on the main body MB. However, the present exemplary embodiment is not limited thereto.

As shown in FIG. 2, the measuring unit 140 may include a current supply 142, a voltage detector 144, and an impedance calculator 146. The voltage detector 144 may include an operation amplifier configured to amplify a voltage between the first output electrode 115 and the second output electrode 120, a filter configured to remove noise, etc. A current is applied by the current supply 142 to the first and second input electrodes 110 and 125, and the voltage is detected by the voltage detector 144 from the first and second output electrodes 115 and 120. The impedance calculator 146 calculates the bio impedance from an input current and the detected voltage.

As shown in FIGS. 3A and 3B, the wearable apparatus 100 for obtaining biological information includes the main body MB and the straps ST. The straps ST are connected to the main body MB and may be placed on a wrist of the subject. The first input electrode 110 and the first output electrode 115 may be arranged on an inner surface STb of any one of the straps ST, and the second output electrode 120 and the second input electrode 125 may be arranged on an outer surface STa of any one of the straps ST.

The first input electrode 110 and the first output electrode 115 are electrodes to be in contact with a wrist of the subject when a user, that is, the subject whose body composition is to be measured, wears the wearable apparatus 100 for obtaining biological information. The first input electrode 110 and the first output electrode 115 may be arranged on locations that allow the first input electrode 110 and the first output electrode 115 to be in contact with the wrist of the subject, and the locations are not limited to the inner surface STb of the straps ST. For example, the first input electrode 110 and the first output electrode 115 may be arranged on an inner surface of the main body MB.

The second output electrode 120 and the second input electrode 125 are electrodes contacting a distal body part corresponding to the other wrist on which the wearable apparatus 100 for obtaining biological information is not placed. The second output electrode 120 and the second input electrode 125 may be arranged on an outer surface of the wearable apparatus 100 for obtaining biological information such that the second output electrode 120 and the second input electrode 125 may be in contact with a body part corresponding to the other wrist. The arrangement locations of the second output electrode 120 and the second input electrode 125 are not limited to the outer surface STa of the straps ST. For example, the second output electrode 120 and the second input electrode 125 may be arranged on the outer surface of the main body MB.

The first input electrode 110 and the first output electrode 115 respectively face the second output electrode 120 and the second input electrode 125, but the present exemplary embodiment is not limited thereto. The first input electrode 110 and the first output electrode 115 may not exactly face the second output electrode 120 and the second input electrode 125. Also, the first input electrode 110, the first output electrode 115, the second input electrode 120, and the second output electrode 125 are arranged to be perpendicular to a lengthwise direction of the straps ST, but a direction in which the first input electrode 110, the first output electrode 115, the second output electrode 120, and the second input electrode 125 are arranged is not limited thereto. The first input electrode 110, the first output electrode 115, the second output electrode 120, and the second input electrode 125 may be arranged in a different direction from the above, for example, in a direction parallel to the lengthwise direction of the straps ST, or other directions.

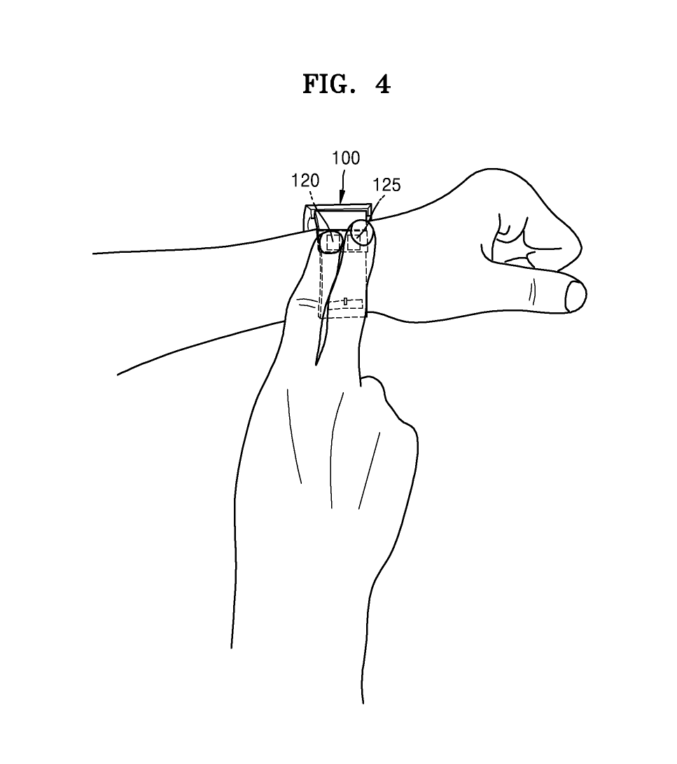

When the subject wears the wearable apparatus 100 on one of his/her wrists in order to measure a bio impedance and the second output electrode 120 and the second input electrode 125 are in contact with a body part corresponding to the other wrist, the subject may be in various positions. The measured bio impedance may vary, depending on the positions of the subject, which will be described with reference to FIGS. 4, 5, and 6A to 6D.

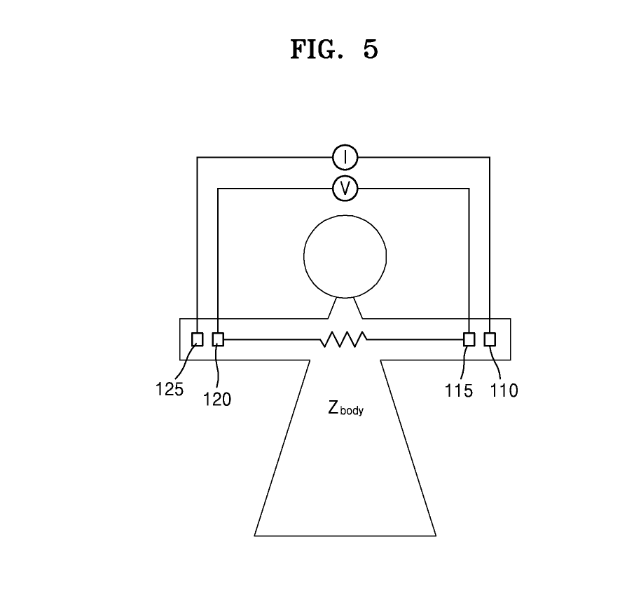

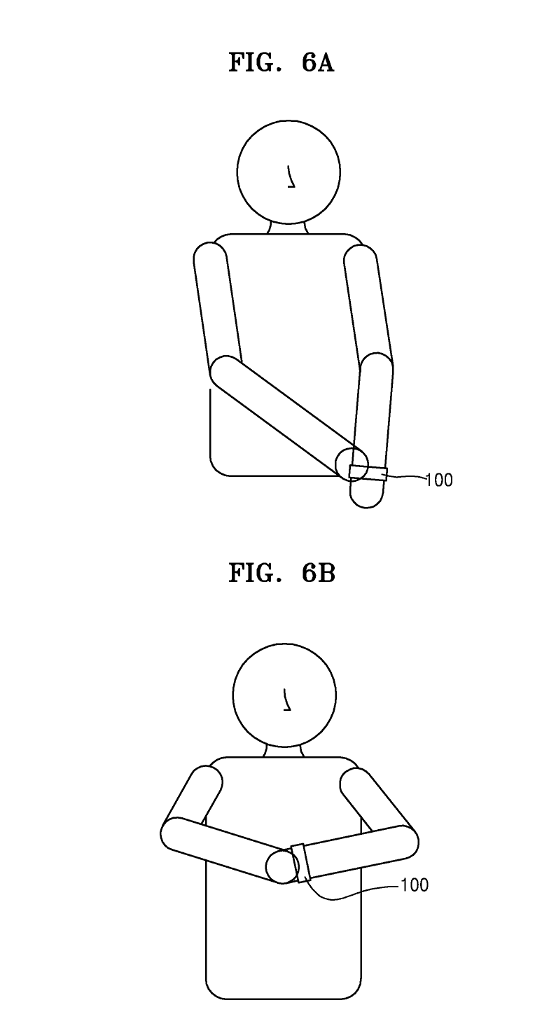

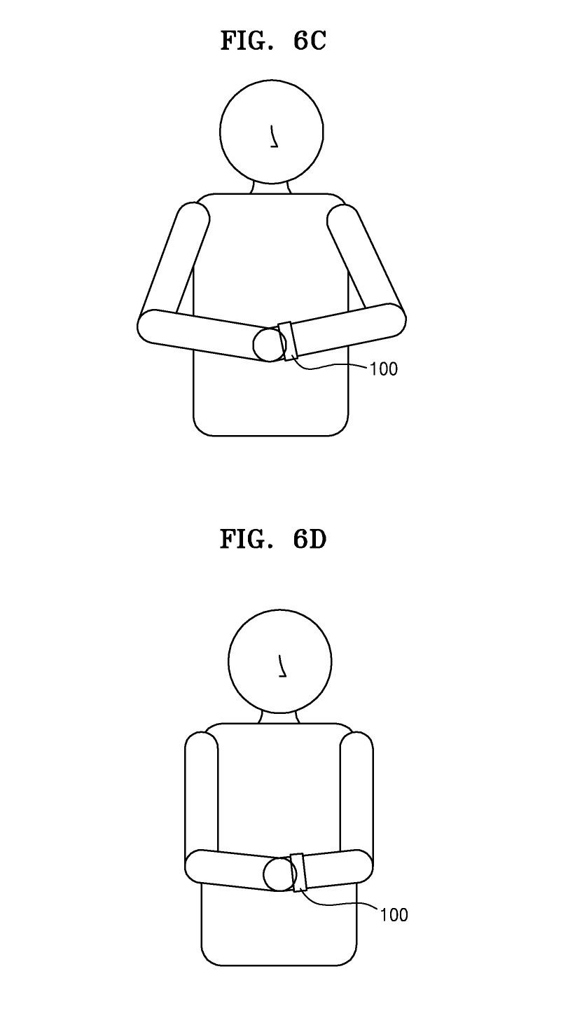

FIG. 4 illustrates a case where a body part comes into contact with an electrode when body composition is measured using the wearable apparatus 100 for obtaining biological information, according to an exemplary embodiment, FIG. 5 schematically illustrates an equivalent circuit when a body part comes into contact with an electrode as illustrated in FIG. 4, and FIGS. 6A to 6D are examples of a variety of certain positions of a subject when the subject comes into contact with an electrode as illustrated in FIG. 4.

As shown in FIG. 4, the subject wears the wearable apparatus 100 for obtaining biological information on a wrist and then may contact the second input electrode 125 and the second output electrode 120 with fingers corresponding to the other wrist.

As shown in FIG. 5, a current is applied to the first and second input electrodes 110 and 125, that is, a closed circuit loop from the first input electrode 110, to a bio impedance Z_{body}, to the second input electrode 125 is formed. The bio impedance Z_{body} may be calculated by detecting a voltage between the first and second output electrodes 115 and 120, and the body composition may be analyzed by using the calculated bio impedance Z_{body}.

The bio impedance Z_{body} may have different values, depending on measuring positions of the subject. Referring to FIGS. 6A to 6D, when the subject wears the wearable apparatus 100 for obtaining biological information on a wrist and contacts the second input electrode 125 and the second output electrode 120 with fingers of the other wrist, the subject may be in various positions. For example, an angle at which both arms are bent, and a distance between both arms and a torso may vary each time, depending on subjects.

When a subject uses an existing large body composition analyzer without wearing the same, the body composition analyzer guides the subject to spread both arms and legs to decrease error factors.

However, while the subject is wearing the wearable apparatus 100 on one of his/her wrists and spreading both of the arms widely, it may be difficult to place his/her fingers of the other wrist on electrodes of the wearable apparatus 100.

In FIG. 6A, it may be understood that the subject spreads both arms as much as possible, but the position is uncomfortable and still has error factors. Measuring positions of FIGS. 6B to 6D are more comfortable than the measuring position of FIG. 6A, but the arms are bent. Thus, the measuring positions of FIGS. 6B to 6D have error factors.

In the present exemplary embodiment, the position detector 150 is included to analyze the body composition by reflecting the error factors. Therefore, the position detector 150 may be configured to detect factors having an influence on measurement values of the bio impedance, for example, an angle at which both arms are bent, distance between both arms and the torso, etc., and calculate correction factors used to correct the measured bio impedance according to the detected measuring positions. Detailed structure and operations of the position detector 150 will be described later with reference to FIGS. 7 to 12.

Referring back to FIG. 1, the remaining components of the wearable apparatus 100 for obtaining biological information will be described.

The analysis unit 160 is configured to analyze body composition of the subject by reflecting the correction factors calculated by the position detector 150 to the bio impedance measured by the measuring unit 140. The body composition may include body fat, characteristics of skin (for example, moisture content), muscle strength, an edema value, or the like.

Various operations used by the measuring unit 140, the position detector 150, and the analysis unit 160 may be stored as programs in the memory 165 and may be executed by a processor. The processor may be hardware for controlling functions and operations of the wearable apparatus 100 for obtaining biological information overall and may control calculation of an impedance in the measuring unit 140, calculation of a correction factor in the position detector 150, and analysis of body composition in the analysis unit 160 by executing the programs stored in the memory 165. In addition, the processor may control the measuring unit 140 to measure the bio impedance and may convert a result regarding the analyzed body composition into image signals in order to display the result on the display 175.

The memory 165 may store programs for operations of the wearable apparatus 100 for obtaining biological information, data necessary for the programs, etc. therein. The memory 165 is a conventional storage medium and may include, for example, a hard disk drive (HDD), read only memory (ROM), random access memory (RAM), flash memory, and a memory card.

The memory 165 may store programs for operations to be performed by the measuring unit 140, the position detector 150, the analysis unit 160, etc. therein. Also, additional data such as an age, weight, gender, etc. may be stored in the memory 165.

The input unit 170 and the display 175 form an interface between the wearable apparatus 100 for obtaining biological information and the subject or a user.

An input for manipulating the wearable apparatus 100 for obtaining biological information may be received via the input unit 170, and a result generated by the analysis unit 160 may be displayed on the display 175.

The input unit 170 may include a button, key pad, switch, dial or touch interface for allowing the subject to directly manipulate the wearable apparatus 100 for obtaining biological information.

The display 175 is a display panel for outputting an analysis result, may include a liquid crystal display (LCD) panel, an organic light-emitting diode (OLED) panel, etc., and may display information regarding a result of analyzing the body composition as an image or in text form. The display 175 may be a touch screen capable of inputting and outputting.

As a user interface, the display 175 may include input/output (I/O) ports for connecting human interface devices (HIDs) and inputting/outputting images.

The communication unit 180 may perform a function for transmitting the analysis result to an external device in a wired or wireless manner. The external device may be, for example, a medical device using analyzed biological information, a printer for printing a result, or a display device for displaying an analysis result. Also, the external device may be a smart phone, a mobile phone, a personal digital assistant (PDA), a laptop, a personal computer (PC), or a mobile or non-mobile computing device, but is not limited thereto.

The communication unit 180 may be connected to the external device in a wired or wireless manner. For example, the communication unit 180 communicates with the external device through a Bluetooth, Bluetooth Low Energy (BLE), Near Field Communication unit (NFC), WLAN (Wi-Fi), Zigbee, infrared data association (IrDA), Wi-Fi Direct (WFD), ultra wideband (UWB), Ant+, and Wi-Fi communication method, but the communication method is not limited thereto.

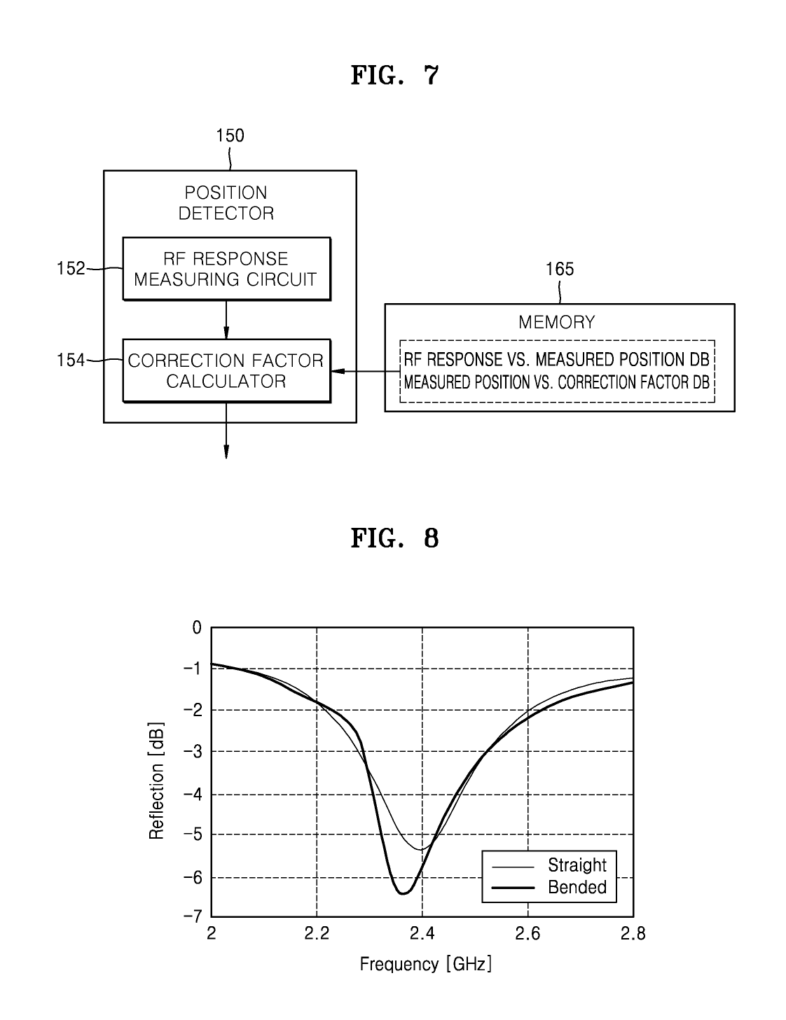

FIG. 7 is a block diagram of an exemplary structure of the position detector 150 included in the wearable apparatus 100 for obtaining biological information, according to an exemplary embodiment, and FIG. 8 is a graph for explaining a radio frequency (RF) response characteristic that changes in the position detector 150, depending on a measuring position of a subject.

The position detector 150 may be configured to detect a measuring position of the subject by analyzing the RF response characteristic and calculate a correction factor based on the detected position of the subject. The position detector 150 may include a circuit configuration used to measure the RF response characteristic and may detect a measuring position of the subject from an antenna included in the circuit configuration and the RF response characteristic that changes according to a relative location of the subject.

FIG. 8 is a computer simulation graph for explaining resonance characteristics that change when arms of a subject who wears a resonator in a band of 2.5 GHz are straight and are bent at a right angle. According to the graph, when the arms functioning as dielectrics are straight or bent, the resonance characteristics of the resonator placed on the wrist of the subject are affected. When the arms are bent, a resonant wavelength band is changed to about 2.36 GHz, and a reflectivity is increased to about 1.4 dB.

The position detector 150 may include an RF response measuring circuit 152 and a correction factor calculator 154. The body of the subject may be considered as a dielectric having high permittivity, and an electric field may change according to a relative arrangement of the body and the antenna. The RF response measuring circuit 152 may detect the change of the electric field to detect a measuring position of the subject. In detail, characteristics such as a resonant frequency, a resonant bandwidth, a power of reflected wave, an impedance, a strength of antenna reception signals, etc. may be measured. The measured characteristics may be used to detect a measuring position of the subject and calculate a correction factor.

The position detector 150 may use the stored data in which a change of the RF response characteristic is quantified depending on a measuring position of the subject. The data may be stored in the memory 165 as a database regarding an RF response and the measuring positions. The measuring position of the subject may be detected by comparing the measured RF response characteristic with the data. Also, the position detector 150 may use the data stored by quantifying a correction factor regarding the measuring position and may calculate the correction factor by using the data. The data may be stored in the memory 165.

As described with reference to FIG. 1, the wearable apparatus 100 for obtaining biological information may further include a wireless communication unit, and in this case, the position detector 150 may share at least some of circuit components forming the wireless communication unit. For example, the position detector 150 may share an antenna used for Bluetooth communication, etc., and accordingly, the number of components, an entire size of a circuit, etc. may be decreased.

The antenna included in the position detector 150 may have a circuit or oval radiation pattern. Also, the radiation pattern of the antenna may be adjusted. For example, the radiation pattern of the antenna may be adjusted overall by including a plurality of antennas having slightly different patterns from each other and a beam switching circuit and by selectively using some of the antennas.

Alternately, a radiation characteristic may be changed by using a reconfigurable antenna. The change of the radiation characteristic may result in the adjustment of a radiation pattern appropriate to detect a position. When the reconfigurable antenna is used, the RF response characteristic according to the measuring position may be more accurately changed, and thus, it may be more advantageous to detect the measuring position.

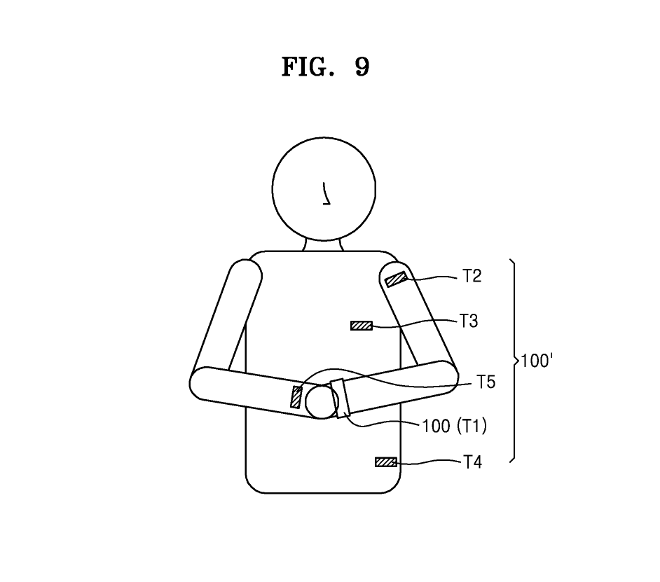

FIG. 9 illustrates a schematic structure of a wearable apparatus 100′ for obtaining biological information according to another exemplary embodiment.

The wearable apparatus 100′ for obtaining biological information further includes one or more RF terminals that are not included in the wearable apparatus 100 for obtaining biological information. In FIG. 9, other than an RF terminal T1 that is installed inside the wearable apparatus 100′ for obtaining biological information, four RF terminals T2, T3, T4, and T5 that are placed on a wrist, on which the wearable apparatus 100′ for obtaining biological information is not placed, and other body parts are illustrated. However, the number or locations of the RF terminals are not limited thereto.

As the RF terminals are included in the wearable apparatus 100′ for obtaining biological information, a measuring position of the subject may be detected in more detail by using a transmission characteristic between two RF terminals, a reflection characteristic of each RF terminal, etc.

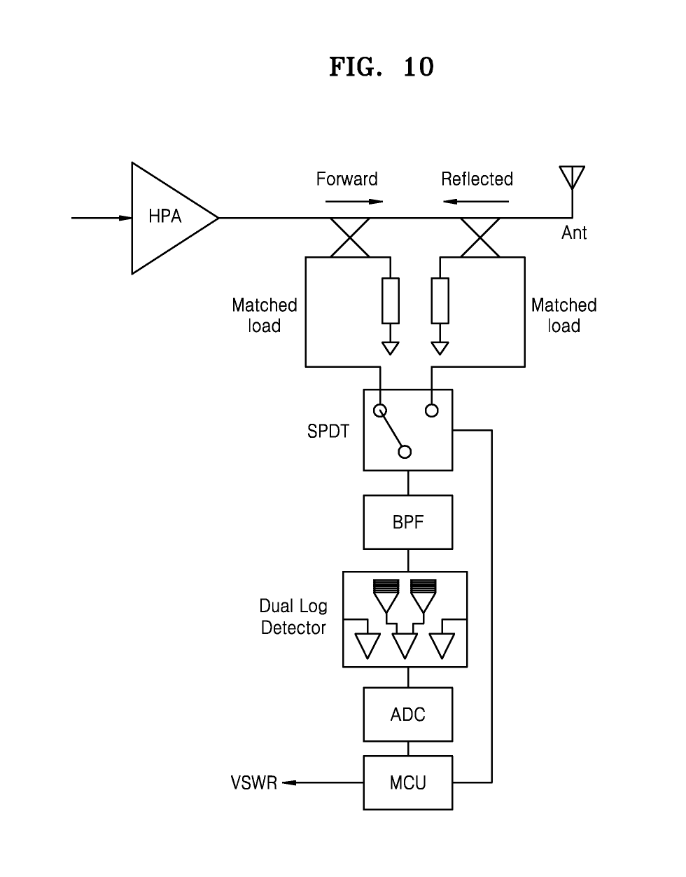

FIG. 10 illustrates an exemplary structure of a circuit that may be used as an RF response characteristic measuring circuit of FIG. 7.

The circuit of FIG. 10 is a circuit for measuring a power of reflected wave. According to an exemplary embodiment, the circuit may include a high power amplifier (HPA), an antenna, matched loads, a single pole double throw (SPDT) switch, a bandpass filter (BPF), a dual log detector, an analog-digital converter (ADC) and a microcontroller (MCU). The circuit may be used to separate a power input to the antenna and a power of the reflected wave from the antenna and detect a difference between the input power and the reflected power by using the dual log detector. A detection result may be output as, for example, a voltage standing wave ratio (VSWR).

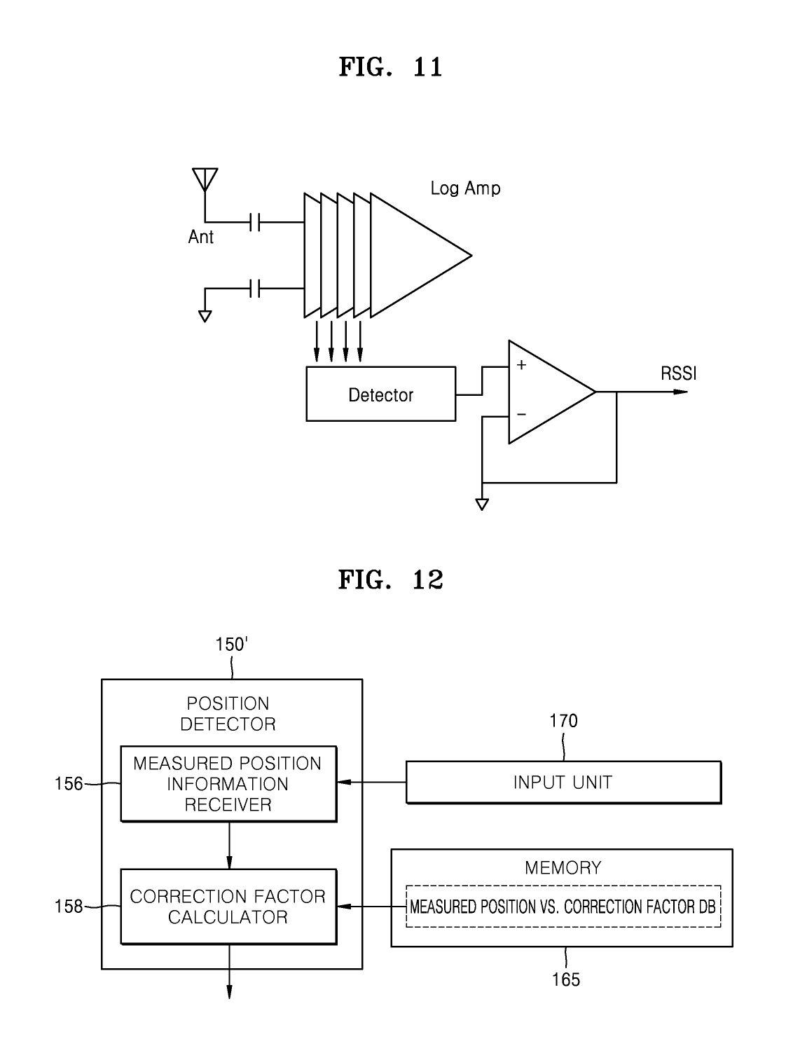

FIG. 11 illustrates an exemplary structure of a circuit that may be used as an RF response characteristic measuring circuit of FIG. 7.

The circuit of FIG. 11 is a received signal strength indication (RSSI) circuit and may be more useful for, for example, the structure of FIG. 9 that further includes additional RF terminals.

The RSSI circuit is generally included in a wireless communication device, and when the wearable apparatus 100 for obtaining biological information is included in a wireless communication device such as a smart watch, the RSSI circuit may be used.

FIG. 12 is a block diagram of an exemplary structure of a position detector 150′ included in a wearable apparatus for obtaining biological information according to another exemplary embodiment.

The wearable apparatus for obtaining biological information of the present exemplary embodiment is configured to use a structure for measuring a bio impedance, that is, a structure including the first and second input electrodes 110 and 125, the first and second output electrodes 115 and 120, and the measuring unit 140 of the wearable apparatus 100 for obtaining biological information of FIG. 1 in order to detect a measuring position of the subject. That is, the position detector 150′ has a different structure from the structure of the position detector 150 of the wearable apparatus 100 for obtaining biological information of FIG. 1.

As described above, values of the bio impedance may vary, depending on the measuring positions as shown in FIGS. 6A to 6D. Therefore, the bio impedance is measured by changing the measuring positions when the subject and a reference regarding the bio impedance are fixed, and thus, a relationship between a measurement value of the bio impedance and the measuring positions may be deducted. Also, a deduction result is quantified as a relation between the measuring positions and a correction factor regarding the measuring positions, and the quantified relation may be stored in a memory as a database and used by the position detector 150′.

The position detector 150′ may include a measuring position information receiver 156 and a correction factor calculator 158. A measuring position of the subject may be input by the input unit 170, and input information is transmitted to the measuring position information receiver 156. The correction factor calculator 158 may calculate a correction factor regarding the measuring position, which is input, from the data stored in the memory 165.

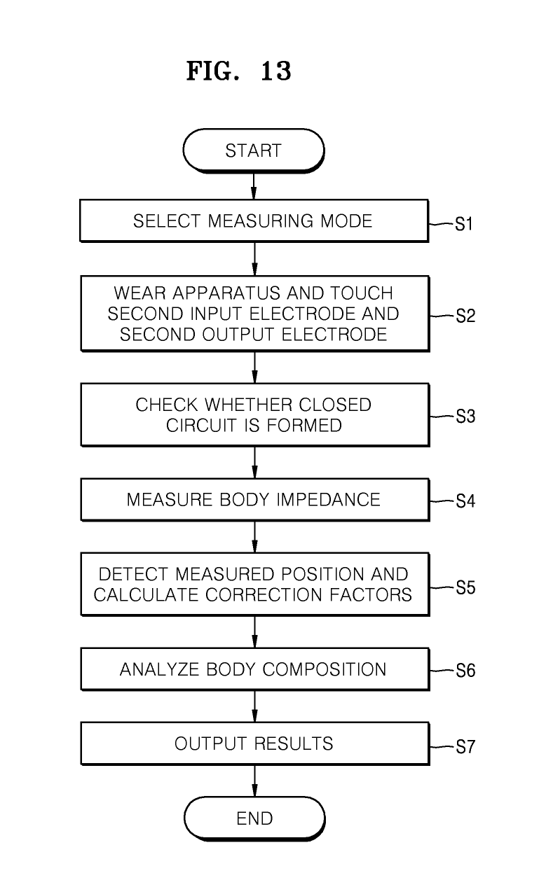

FIG. 13 is a schematic flowchart for explaining a method of measuring body composition according to an exemplary embodiment.

The subject wears a wearable apparatus for obtaining biological information on a left or right wrist in order to measure body composition.

In operation S1 in which a measurement mode is selected, a height, weight, etc. of the subject may be input. Also, according to a method of calculating a correction factor, a measuring position may be input if necessary. The measuring position may be input by selecting one of a plurality of menus that are configured by properly combining, for example, an angle at which both arms are bent, a distance between both arms and a torso, and a distance of wrists and the torso.

In operation S2, the wearable apparatus may be placed on the left or right wrist of the subject and a second input electrode and a second output electrode of the wearable apparatus may be in contact with a body part corresponding to the other one of the left and right wrist on which the wearable apparatus is not placed. The second input electrode and the second output electrode may refer to electrodes that are not placed in contact with the wrist on which the wearable apparatus for obtaining biological information is placed and may be, for example, the second input electrode 125 and the second output electrode 120 of the wearable apparatus 100 for obtaining biological information of FIG. 1.

The order of operations S1 and S2 is exemplary, and the subject may wear the wearable apparatus on one wrist and then select a measurement mode and contact electrodes with a body part corresponding to the other wrist.

In operation S3, the wearable apparatus determines whether a closed circuit is formed. If the wearable apparatus confirms that the closed circuit is formed, the wearable apparatus may measure a bio impedance in operation S4. Operations S3 and S4 may be performed by applying a current to input electrodes and detecting a voltage from output electrodes. When the electrodes and a body part do not properly come into contact with each other, the closed circuit is not formed, and significant measurement values are not detected. In this case, it is required to check a contact state first and then to perform measurement.

In operation S5, a measuring position is detected, and a correction factor regarding the measuring position is calculated.

Operation S5 may be performed by measuring an RF response characteristic and calculating a correction factor regarding the RF response characteristic. Alternately, in accordance with the structure of FIG. 12, a correction factor corresponding to the measuring position, which is input, may be calculated by comparing the correction factor corresponding to the measuring position with a correction factor stored in accordance with a measuring position.

A performance order of operations S4 and S5 may be reversed.

In operation S6, the body composition is analyzed by reflecting the calculated correction factor to the measured bio impedance.

An analysis result of the body composition is output as an image or in text form in operation S7. Outputting a result may be performed by displaying the result on a display included in the wearable apparatus for obtaining biological information, transmitting the result to another device so as to display the result on a display included in the other device, or outputting the result via a printer.

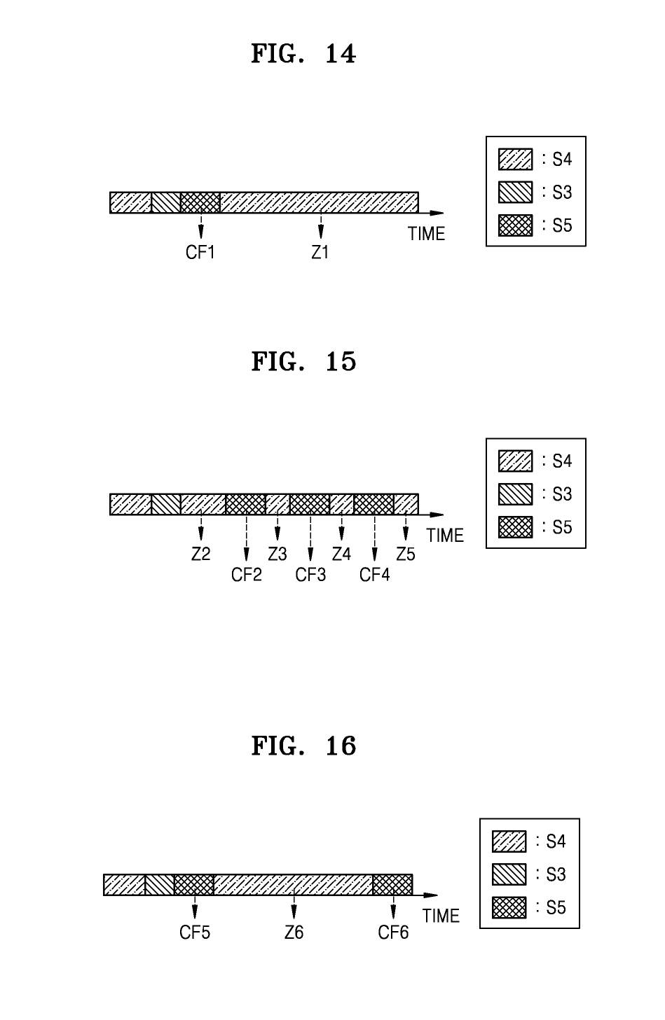

FIGS. 14 to 17 are schematic flowcharts for explaining a method of measuring body composition according to another exemplary embodiment. In FIGS. 14 to 17, operation S3 in which whether a closed circuit is formed is confirmed, operation S4 in which a bio impedance is measured, and operation S5 in which a measuring position is detected and a correction factor is calculated are performed in a different order from the above-described order or repeated.

Referring to FIG. 14, after confirming that a closed circuit is formed in operation S3, the wearable apparatus firstly performs operation S5, in which a measuring position is detected and a correction factor is calculated, and then performs operation S4, in which a bio impedance is measured. The body composition is analyzed from the correction factor CF1 calculated in operation S5, in which a measuring position is detected and a correction factor is calculated, and the bio impedance Z1 measured in operation S4 in which a bio impedance is measured.

Referring to FIG. 15, after confirming that a closed circuit is formed in operation S3, the wearable apparatus performs operation S4, in which a bio impedance is measured, and thereafter performs operation S5, in which a measuring position is detected and a correction factor is calculated. The wearable apparatus alternately repeats operations S4 and S5 more than twice in the order described above. The repetition is for taking into account a possible change of the measuring position being measured. The body composition may be analyzed from bio impedances Z2, Z3, Z4, and Z5 that are measured in respective operations and correction factors CF2, CF3, and CF4 that are calculated in respective operations. Depending on measuring positions of the subject, some of the bio impedances Z2, Z3, Z4, and Z5 and the correction factors CF2, CF3, and CF4 may have identical values. In this case, the body composition may be analyzed from the remaining bio impedances Z2, Z3, Z4, and Z5 and the correction factors CF2, CF3, and CF4 which have different values.

Referring to FIG. 16, after confirming that a closed circuit is formed in operation S3, the wearable apparatus performs operation S5, in which a measuring position is detected and a correction factor is calculated, and then performs operation S4, in which a bio impedance is measured. In addition, operation S5, in which a measuring position is detected and a correction factor is calculated, is repeated. The body composition may be analyzed from correction factors CF5 and CF6 and a bio impedance Z6. When the correction factors CF5 and CF6 are identical because a measuring position of the subject is maintained, any one of the correction factors CF5 and CF6 may be used to analyze the body composition.

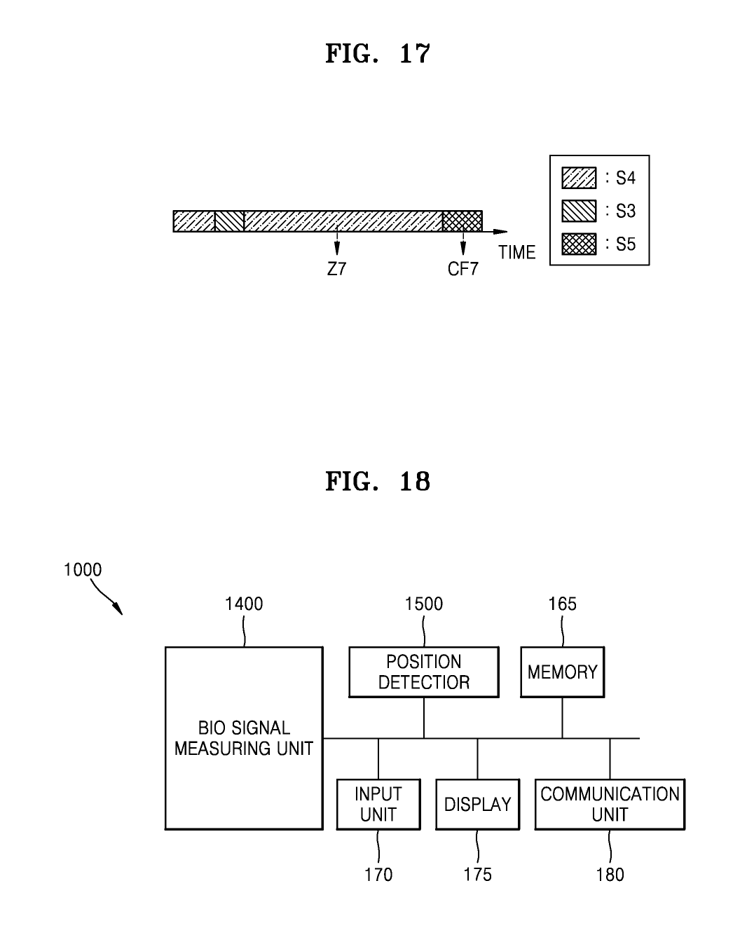

Referring to FIG. 17, after confirming that a closed circuit is formed in operation S3, the wearable apparatus performs operation S4, in which a bio impedance is measured is performed, and then performs operation S5, in which a measuring position is detected and a correction factor is calculated. The body composition may be analyzed from a correction factor CF7 and a bio impedance Z7.

As another modified example, operation S4, in which a bio impedance is measured, and operation S5, in which a measuring position is detected and a correction factor is calculated, may be simultaneously measured. The simultaneous performance does not mean that a start and end of operations are exactly the same but means that operations may temporally overlap.

The detection of the measuring position and the calculation of the correction factor regarding the measuring position are performed by an apparatus for measuring body composition, the apparatus configured to measure a bio impedance and analyze the body composition. However, the inventive concept is not limited thereto.

Various types of wearable apparatuses for obtaining biological information may include a position detector having the above-described functions.

FIG. 18 is a block diagram of a schematic structure of a wearable apparatus 1000 for obtaining biological information according to another exemplary embodiment. The wearable apparatus 1000 for obtaining biological information may include a bio signal measuring unit 1400 configured to measure and analyze bio signals of the subject and a position detector 1500 configured to detect a position of the subject wearing the wearable apparatus 1000 for obtaining biological information and calculate correction factors regarding the bio signals in response to the detection of the position. Also, the wearable apparatus 1000 for obtaining biological information may further include a memory 165, a display 175, an input unit 170, a communication unit 180, or the like. Similar to the illustrations of FIGS. 3A and 3B, the wearable apparatus 1000 for obtaining biological information may be of a watch-type, but types of the wearable apparatus 1000 for obtaining biological information are not limited thereto.

As described above, the position detector 1500 includes a circuit for measuring RF response characteristics, and a position of the subject may be detected from RF response characteristics that change according to a relative location between the subject wearing the wearable apparatus 1000 for obtaining biological information and an antenna of the circuit. That is, RF response characteristics, which are measured from an RF response measuring circuit, may differ according to a position of the subject wearing the wearable apparatus 1000 for obtaining biological information, for example, a particular position of a body part on which the wearable apparatus 1000 for obtaining biological information is placed, and the position of the subject may be detected from the RF characteristics. Furthermore, if the wearable apparatus 1000 for obtaining biological information includes a wireless communication unit, the RF response measuring circuit may share some components of a circuit included in the wireless communication unit, and thus, a circuit having a decreased number of components may be configured.

Alternately, the position detector 1500 includes a bio impedance measuring circuit and thus may detect a position of the subject from the bio impedance of the subject wearing the wearable apparatus 1000 for obtaining biological information.

The bio signal measuring unit 1400 may measure and analyze bio signals regarding a blood pressure of the subject. For example, the bio signal measuring unit 1400 may include a sensor configured to irradiate light onto a radial artery of the subject and detect light reflected thereform, and an analysis unit configured to detect photoplethysmograph (PPG) signals from detected light signals and analyze a blood pressure.

A relative location between the heart and a location where the light signals are detected, that is, a relative location between the heart and a location where the sensor is disposed, for example, between the heart and a wrist, may change due to the position of the subject.

A height difference between the heart and a detection location (for example, a wrist) may cause a change in the blood pressure that occurs due to gravity. Thus, a waveform of bio signals to be used to analyze the blood pressure may change according to the relative location between the heart and the detection location. Therefore, a database DB regarding RF response information generated from an angle of arms, a distance between a torso and the arms, a distance between the torso and the wrists, etc. and a relative height of the arms according to the RF response information is formed, and the formed DB may be used as correction factors that are used to analyze the blood pressure based on the measured bio signals.

The wearable apparatus for obtaining biological information analyzes the measuring position of the subject and uses the same to analyze the biological information, and thus, the accuracy of the biological information measurement may be increased.

Also, since the subject is not required to be in a particular position while the position of the subject is being measured, user convenience is increased.

The foregoing exemplary embodiments and advantages are merely exemplary and are not to be construed as limiting. The present teaching can be readily applied to other types of apparatuses. Also, the description of the exemplary embodiments is intended to be illustrative, and not to limit the scope of the claims, and many alternatives, modifications, and variations will be apparent to those skilled in the art.

What is claimed is:

1. A wearable apparatus for obtaining biological information, the wearable apparatus comprising:
a bio signal measuring unit configured to measure a bio signal of a subject; and
a position detector configured to detect a position of the subject wearing the wearable apparatus and determine correction factors with regard to the measured bio signal in response to the detected position.

2. The wearable apparatus of claim 1, wherein the position detector comprises a circuit configured to measure a radio frequency (RF) response characteristic, and the position detector is further configured to detect the position of the subject from the measured RF response characteristic that changes according to a relative location between the subject wearing the wearable apparatus and an antenna included in the circuit.

3. The wearable apparatus of claim 2, further comprising a wireless communication unit,
wherein the position detector is further configured to share at least one of circuit components forming the wireless communication unit.

4. The wearable apparatus of claim 1, wherein the position detector comprises a circuit configured to measure a bio impedance of the subject, and the position detector is further configured to detect the position of the subject from the measured bio impedance.

5. The wearable apparatus of claim 1, wherein the bio signal measuring unit comprises:
a first input electrode and a first output electrode arranged to be in contact with one wrist of the subject;
a second input electrode and a second output electrode arranged to be in contact with a body part corresponding to the other wrist of the subject;
a measuring unit configured to measure a bio impedance of the subject by applying a current to the first and second input electrodes and detecting a voltage from the first and second output electrodes; and
an analysis unit configured to analyze a body composition of the subject by reflecting the correction factors determined by the position detector to the bio impedance measured by the measuring unit.

6. The wearable apparatus of claim 5, wherein the position detector is further configured to detect at least one of an angle at which an arm of the subject is bent and a distance between the wrists and a torso of the subject as a measuring position.

7. The wearable apparatus of claim 5, wherein the position detector comprises a circuit for measuring a radio frequency (RF) response characteristic and is further configured to detect a measuring position of the subject from the RF response characteristic that changes according to a relative location between the subject and an antenna included in the circuit.

8. The wearable apparatus of claim 7, wherein the position detector is further configured to use data stored by quantifying a degree to which the RF response characteristic changes according to the measuring position of the subject and detect the measuring position of the subject by comparing the data with the measured RF response characteristic.

9. The wearable apparatus of claim 8, wherein the position detector is configured to use data stored by quantifying a correction factor regarding the measuring position of the subject and determine the correction factor by comparing the measuring position of the subject with the data stored by quantifying the correction factor.

10. The wearable apparatus of claim 7, wherein the position detector further comprises at least one RF terminal to be placed on a body part of the subject, other than the wrist on which the wearable apparatus is placed.

11. The wearable apparatus of claim 7, wherein the antenna has a circular or oval radiation pattern.

12. The wearable apparatus of claim 7, wherein the position detector is further configured to receive information regarding the measuring position of the subject, quantify a correction factor regarding the measuring position of the subject by using data stored in the wearable apparatus, and determine the correction factor by comparing the received information regarding the measuring position of the subject with the stored data.

13. The wearable apparatus of claim 1, wherein the bio signal indicates a blood pressure of the subject.

14. A method of obtaining biological information by using a wearable apparatus including a first input electrode, a second input electrode, a first output electrode, and a second output electrode, the method comprising:
measuring a bio impedance of a subject by applying a current to the first input electrode and the second input electrode and detecting a voltage from the first output electrode and the second output electrode, the first input electrode and the first output electrode being in contact with one wrist of a subject, and the second input electrode and the second output electrode being in contact with a body part of the other wrist;
detecting a measuring position of the subject;
determining a correction factor regarding the measuring position; and
analyzing the body composition of the subject by reflecting the correction factor to the bio impedance measured by the measuring unit.

15. The method of claim 14, further comprising checking whether each of the first input electrode, the second input electrode, the first output electrode, and the second output electrode of the wearable apparatus is placed in contact with the subject.

16. The method of claim 14, wherein the measuring the bio impedance is performed after the detecting the measuring position and the determining the correction factor are performed.

17. The method of claim 14, further comprising repeating the detecting the measuring position and the determining the correction factor after the measuring the bio impedance.

18. The method of claim 14, wherein the detecting the measuring position and the determining the correction factor are performed after the measuring the bio impedance.

19. The method of claim 14, wherein the measuring the bio impedance, the detecting the measuring position, and the determining the correction factor are respectively repeated at least twice.

20. The method of claim 14, wherein the measuring the bio impedance, the detecting the measuring position, and the determining the correction factor are simultaneously performed.
